(19) United States
(12) Patent Application Publication
(10) Pub. No.: US 2026/0216444 A1
(43) Pub. Date: Jul. 30, 2026

(54) MULTI-DOSE SYRINGE

(71) Applicant: Stephen Terence DUNNE, Porto (PT)

(72) Inventor: Stephen Terence DUNNE, Porto (PT)

(21) Appl. No.: 19/147,295

(22) PCT Filed: Jan. 5, 2024
(86) PCT No.: PCT/EP2024/050218

§ 371 Date: Jul. 10, 2025

(30) Foreign Application Priority Data
Jan. 12, 2023 (EP) 23151381.3

(51) Int. Cl. A61M 5/315; A61M 5/00; A61M 5/24; A61M 5/31

(52) U.S. Cl. CPC A61M 5/31595; A61M 5/002; A61M 5/2429; A61M 5/3137; A61M 5/31505; A61M 2005/31508

(57) ABSTRACT

An accurate-dose and/or multi-dose syringe comprising an outer casing or syringe housing with finger holds at the proximal end and a drug delivery port at the distal end capable of firmly holding a drug container having a generally tubular glass or plastic barrel with drug delivery means at the distal end of the barrel and a stopper/piston disposed close to the proximal end of the barrel to contain the medicament or other formulation between the stopper/piston and the drug delivery means.

The invention relates to a syringe system, in particular an accurate-dose and/or a multi-dose manually operated syringe system.

Glass and plastic pre-filled syringes or ready-to-fill syringes are supplied for use with the drug formulation already in the syringe and ready to use. There are two formats. The first has a luer connection for attaching a hypodermic needle. The second format, the staked needle syringe, has the needle already attached. Both formats need to be lubricated with silicone oil to facilitate stopper or piston travel.

Both have advantages and disadvantages. The principal advantage of the Glass Luer type is that the silicone lubrication can be baked on giving it superior lubrication and the choice of needle is unlimited. The biggest disadvantage is that the needle needs to be attached by the user which can lead to user errors and even loss of sterility.

Glass staked needle syringes come with the needle attached and held in place which is its biggest advantage. The disadvantages are that the needle, epoxy glue and residue tungsten from its manufacture are in contact with the drug formulation during storage and that the needle tip can be damaged or blunted by the needle shield because the needle is embedded into the rubber shield to seal it. Additionally, silicone lubrication oil cannot be baked on because of the epoxy glue so staked needle pre-filled syringes must rely on inferior sprayed on silicone where the silicone can migrate during storage into the dug causing drug agglomeration or migrate between areas on the glass surface leaving areas unlubricated.

Additionally, staked needle pre-filled syringes have limited needle options and are available only in ready to fill or ready to use formats. These are expensive compared to bulk formats. These formats also need dedicated filling lines. Staked needle pre-filled syringes also cannot be terminally gas sterilized. This is because the needle shied is gas permeable to allow gas during the original sterilization before filling to sterilize the needle. Once filled with drug it cannot be gas sterilized because gas could inter the container via the needle.

Vaccine drug containers are usually multi dose vials or single dose pre-filled syringes. When supplied in vials the individual doses or vaccines need to be transferred from the vial to individual disposable syringes. This is time consuming and expensive and prone to clinical errors including filling with the wrong dose and loss of sterility. If they are supplied in pre-filled syringes each individual dose needs its own drug container and need to be individually filled on a filling line and often cold stored. Vials have the advantage that few filling lines or filling heads are required. Filling capacity is often a problem. Often also staked needle pre-filled syringes cannot be used for stability reasons so luer versions are used where the user attaches the injection needle.

An alternative to pre-filled syringes are cartridges that comprise of a glass or plastic barrel, a pierceable septum held by a crimped metal ferrule at one end and a stopper/piston at the other. A septum piercing needle or cannula is used to perforate or pierce the septum making a fluid pathway between the cartridge drug contents and the injection needle or connecting port. Such arrangements are typically used in pen injectors such as insulin pens and cartridge-based dentistry syringes.

Cartridges are available in both ready to fill and much cheaper bulk formats. They can be filled like syringes by capping, filing, and stoppering or bubble free by stoppering, filling and caping.

It is an object of the present invention to provide a multi dose syringe that can be used with various drug containers including cartridges or pre-filled syringes. The present invention both minimizes drug container numbers, storage capacity and filing capacity in comparison to single dose pre-filled syringes and reduces clinical errors relative to the use of vials and disposable syringes.

In accordance with the invention, this objective is achieved with a syringe system as defined in claim 1.

Preferred embodiments of the invention are defined in the dependent claims.

In particular, a syringe system in accordance with the present invention comprises of an:
an outer casing or syringe housing with finger pads or flange or finger rings at the proximal end capable of firmly holding;
a drug container with drug delivery means at the distal end of the barrel and a stopper/piston disposed near the proximal end of the barrel to contain the medicament or other formulation between the distal and proximal ends,
a plunger rod with finger pad at one end and in communication but not attached to the cartridge stopper at the other end where the plunger rod has two or more axial grooves and two or more radial grooves in communication with one another and in engagement with a fixed pin or engagement member connected to the syringe housing and where the pin travels relative to the grooves,
and where the plunger rod is free to rotate relative to the stopper.

In other words, in accordance with one aspect of the invention a guiding and engagement system is provided for the ensemble of plunger and syringe housing, determining the potential movement or displacement of plunger relative to the syringe housing. This guiding and engagement system in an aspect of the invention comprises a number of guiding grooves (in particular, both a number of radial grooves and a number of axial grooves) being in communication with one another which may be positioned either on the outer surface of the plunger or on the inner surface of the syringe housing. Corresponding to the guiding grooves, the guiding and engagement system comprises a pin or engagement member, intended to be slidingly moved in the guiding grooves and positioned at the other of plunger and syringe housing, respectively. By this system, with the pin or engagement member being in engagement with the set of grooves, the pin travels within and relative to the grooves as the user rotates and pushes the plunger rod in the syringe housing. Due to the engagement of pin in the grooves, relative motion of plunger and housing is only possible to the degree allowed by the travelling of the pin in the grooves, and therefore the system of grooves communicating with one another defines the possible motions of the plunger relative to the syringe housing. In consequence, as long as the pin is positioned in a segment of an axial groove, only axial movement of the plunger relative to the housing is allowed and possible. In contrast, as the pin is positioned in a segment of a radial groove, only rotational movement of the plunger relative to the housing is possible.

Therefore, by proper combination and sequence of the groove system provided, certain sequences of motions of the plunger within the housing may be predefined. In one preferred embodiment of the invention, a first axial groove, in which the pin is introduced first when the plunger is inserted into the syringe housing, may be assigned for priming the syringe. Since the length of the respective axial groove determines how far the plunger is allowed to be moved axially before the subsequent, radial groove is entered, the length of the first axial groove in one aspect of the invention is chosen such that the appropriate axial movement of the plunger in order to prime the syringe is allowed.

Upon entering of the pin into the crossover section of the first axial groove and the subsequent radial groove, further axial displacement of the plunger in the syringe housing is impeded. Rather, the pin can then only be moved further in rotational direction of the radial groove, i. e. by rotating the plunger in the syringe housing, until the next crossover to the subsequent axial groove is reached. From there on, further axial displacement of the plunger in the syringe housing is allowed until the pin reaches the next radial groove. Accordingly, axial displacement of the plunger again is limited by the length of the respective axiual groove. In accordance with one aspect of the invention, the length of this respective axial segment is chosen such that a precise dose of liquid formulation is delivered via the drug delivery port. In a preferred embodiment, a number of subsequent radial and axial grooves may be provided in order to allow for a sequence of several well-defined axial displacements of the plunger in the syringe housing each with exactly defined dose delivered, interrupted by the intermediate radial sections. Therefore, a reliable delivery system for multi-dose injections with high precision is achievable.

In a preferred embodiment, the pin or engagement member is biased against the grooves and the grooves have a profile that prevents the user reversing the plunger rod travel.

Priming of the device before the first injection and/or in between injections is also possible with further radial and axial grooves in between the injection grooves. Alternatively, the radial grooves may be angled in the direction of travel so that when the user rotates the plunger as well as the relative radial travel of the pin there is also some relative axial travel.

This may be advantageous as priming the device in between injections ensures that some drug formulation primes any new needle prior to the injection.

The axial grooves may be of different lengths for different dose volumes.

The rotational travel of the radial grooves is preferrably 180 degrees to facilitate the molding of the plunger rod in plastic although other travel distances are possible.

The drug container having a generally tubular glass or plastic barrel may be a cartridge with a pierceable seal or septum or pre-filled syringe with a staked injection needle or luer connector at the distal end of the barrel.

When used with a cartridge either;
a septum needle may be rigidly held in the syringe housing and is in fluid communication with an injection needle or a luer slip or luer lock projecting from the distal end of the syringe housing and where the cartridge is held axially in a first position and is prevented from substantially moving unaided axially forwards within the syringe housing,
or a threaded fitting including means to connect an injection needle and septum needle combination projecting from the distal end of the syringe housing and where the cartridge is held axially in position and is prevented from moving axially within the syringe housing.

When used with a staked needle pre-filled syringe either;
the pre-filled syringe injection needle projects from the distal end of the syringe housing and the pre-filled syringe is held axially in position and is prevented from moving axially within the syringe housing.
and when used with a pre-filled syringe with a luer connection;
the pre-filled syringe luer connection projects from the distal end of the syringe housing and the cartridge is held axially in position and is prevented from moving axially within the syringe housing.

Note that if the injection needle is included the syringes can generally only be used sequentially on one subject or patient. If the device has a luer or threaded connection the needle may be replaced after each dose and used with multiple patients.

The plunger rod axial grooves may be of different lengths to deliver different sized doses.

The first groove may be a radial groove that prevents inadvertent axial plunger movement before the user is ready to use the syringe and during transport and handling and before, where required, the needle is attached and especially where the device is a combination product with the drug container is installed before sterilization. In this case the plunger rod is supplied to the user in place in the devise and the pin is placed after the rod is installed.

Alternatively, the plunger rod may be supplied separately and inserted by the user after the cartridge or PFS is in place. In this case the device may have an additional entry or guiding axial groove terminating at the rod end and a subsequent radial groove placed before the priming or first dose axial groove in the order that the plunger rod may be inserted and rotated by the user before use and remain in a stable position. In this case the needle or line is best attached, where required, before the rod is inserted.

To use the syringe with the drug container and plunger rod in place with the syringe ready for priming;
with a cartridge;
with the injection needle in place and where the housing has a rigidly held septum needle and where the cartridge is free to be moved by the user the user pushes the plunger rod wherein firstly the cartridge is moved axially forward to a second position wherein the cartridge septum is perforated by the septum needle establishing fluid communication between the cartridge contents and the injection needle and further, if required, further pushes the plunger rod priming and expelling any air bubble;
or where the cartridge is fixed in position and after the user attaches a double ended needle where the cartridge septum is perforated by the septum needle establishing fluid communication between the cartridge contents and the injection needle the user pushes the plunger rod wherein priming takes place expelling any air bubble;
with a pre-filled syringe with the injection needle in place and plunger rod ready for priming the user pushes the plunger rod wherein priming takes place expelling any air bubble.

Once priming is accomplished the user further twists or rotates and the pushes the plunger rod to deliver a precise dose of drug formulation. This can be repeated multi times until all the doses are delivered. Priming might not be required and if so the first push of the plunger rod delivers the first injection dose.

When the drug container is a cartridge, the cartridge needs to held at its distal end in the housing either by the cartridge shoulder or by its cap. It is important that the user can view the drug solution before using to check for possible abnormalities such as particles, color, cloudiness etc. For this end the syringe housing may have viewing windows.

In the case of pre-filled syringes, the pre-filled syringe may be held by the shoulder at its distal end like a cartridge or by its flanges at its proximal end, in which case most of the syringe barrel can be exposed beyond a short syringe housing facilitating the viewing of the drug contents.

Where possible the user may replace the needle after each dose is delivered.

The cartridge or pre-filled syringe may be inserted before sterilization or by the user.

The cartridge or pre-filled syringe maybe be inserted axially or radially. When inserted axially the user attaches or positions the plunger rod after inserting the cartridge. When inserted radially or sideways the plunger rod may already be in place.

The invention is generally a single use device where one container is used either supplied installed or inserted by the user. Some embodiments may be re-usable where more than one container can be used and in particular in the case of a cartridge base device where the septum needle is replaced along with the injection needle or in the case of a pre-filled syringe-based device where the needle is attached by the user. In both cases the drug containers are inserted by the user.

With a movable cartridge the cartridge may be held in the first position by a deformable or movable stop on the cartridge shoulder or cap to prevent the cartridge moving forward during transport or handling or after insertion by the user. The cartridge may be held or further held in place by friction or interference fit. The device may have a locking mechanism that holds the cartridge in place before use. If the device has a locking mechanism this needs to be unlocked so that the cartridge can be pushed to the second position by pressing the plunger rod. The lock may hold the deformable stops in the closed position that are released by activating a switch or button or other means such as removing a cap.

The device when used with a cartridge may include a cap to cover the injection needle or luer or other connection prior to use. In the case of a movable cartridge the cap may be used to lock the cartridge at the first position before use. This locking feature may be part of the cap or the housing in which case the lock is held in the locked position by the cap.

The drug container may be filled with or without an air or gas bubble. This can be expelled during priming before the first injection.

The drug container may be a double chamber or a dual chamber container. In this case the first axial movement is additionally used to mix the contents of the two chambers while the second axial movement may be used to prime and expel any air in the container. When the drug container is a cartridge and in particular a moving cartridge the first axial movement also moves the cartridge forwards wherein the septum is perforated by the septum needle before mixing takes place. The drug double chamber container contents may be a liquid and a dry powder or two liquids.

The whole assembly including with or without the drug container may be terminally sterilized in a blister or other outer packaging. Depending on the drug type and packaging and device materials the system can be sterilized using moist heat (steam), dry heat, gamma radiation or other radiation, ethylene oxide gas, vaporized hydrogen peroxide, chlorine dioxide gas, vaporized peracetic acid and nitrogen dioxide. When terminally sterilized with the drug container in place the terminal sterilization may be adjusted to only sterilize the outside surfaces of the syringe and the drug container. However, it can also be adjusted and be used to additionally sterilize the drug contents themselves if required.

If terminally sterilized a staked needle pre-filled syringe may not be used. Either a luer pre-filled syringe is used or a cartridge.

In another aspect of the invention, which is considered independently inventive, if a cartridge is used with a fixed needle attached to the housing in communication with a septum needle the whole assembly may be terminally sterilized in a blister. This has the great advantage in that after removing from the blister the device is ready to use. This is not possible with staked-needle pre-filled syringes as they cannot be terminally sterilized and with luer pre-filled syringes where a needle needs to be attached after removing from blister with consequential possible loss of sterility.

The drug container barrel may be made of glass or plastic such as COC or COP. The syringe system may be adapted for subcutaneous, intramuscular, intradermal, ophthalmologic, intravascular, intravenous or any other type of injections.

The current invention may be used to deliver or inject any liquid into any body including human or animal for any purpose. In one aspect of the invention, which is considered independently inventive, the syringe may by intended for being used to apply Botox to a patient. For this application, preferably the small diameter 0.5 ml pre-filled syringe, either staked needle or luer version, may be used or a small diameter cartridge may be used. The small diameter would give sufficient stroke length for feedback. The doses are around 50 microliters, and the same needle may be used for one injection sequence. If a cartridge is used with a fixed needle this can be terminally sterilized with the needle in place which is a great advantage.

In another aspect of the invention, which is considered independently inventive, the syringe may by intended for ocular, intravitreal or intraocular injections where accuracy of dose is very important. For this application, preferably the small diameter 0.5 ml pre-filled syringe, either staked needle or luer version, may be used. The small diameter would give sufficient stroke length for feedback as the dose is around 50 microliters. For this application it is usual to give one injection with first priming the device to expel air in the system including the needle so in this application the first axial stroke would be the priming stroke and the second the very precise injection stroke. Another big advantage of the present invention is that the user cannot inadvertently reverse the plunger rod travel eliminating the chance of air being sucked into the needle and injected into the eye. Alternatively a small diameter cartridge may be used with a fixed needle that can be terminally sterilized with the needle in place which is a great advantage.

In particular the use of the invention in pre-filled syringes (“PFS”s) may be considered highly beneficial, thereby expanding the scope of possible uses for the Multi-Dose concepts. PFSs are available in smaller diameters than cartridges which increases the stroke length for a given injection and thus makes efficient use of the benefits of the present invention. In particular, current intravitreal injections use 0.5 ml luer PFSs and are preferably terminally Sterilised.

The invention is also ideally suited for multiple self-injections at home either for sequential multi-injections or for daily injections where a new needle can be used for each injection. This has advantages over the use of vials and disposable syringes and over luer pre-filled syringes as fewer drug containers and storage capacity is needed.

The invention is also ideally suited for vaccines where a new needle can be used for each injection. This has advantages over the use of vials and disposable syringes as it greatly reduces potential clinical errors and over pre-filled syringes as fewer drug containers and storage capacity is needed.

The invention may have a dose counter and plunger rod indicators to inform the user of the dose number and the next step. Numbers and arrows and other symbols can be printed or molded on the plunger rod for this purpose. In addition, it can be arranged so the numbers and arrows are clearly seen through a window in the syringe housing.

Environmentally friendly materials may be used to manufacture all or some parts of the syringe system including wood-based materials or bio-degradable plastics.

The current invention is a great improvement over the use of vials and disposable syringes as far fewer user steps are required with less likelihood of clinical errors. It also has advantages over individual dose pre-filled syringes as fewer drug containers and container fillings are required. Additionally, when a cartridge is used, the drug is only in contact with the barrel material, glass or plastic and an elastomer-ic material. This is the same as with storage in a vial. Additionally, the cartridge may have the superior baked-on silicone and the syringe assembly with the cartridge may be terminally sterilized with a needle attached.

The current invention is a great improvement over existing state of the art multi-dose deliver systems as it is of a simpler construction. By not attaching the plunger rod to the stopper the plunger rod can be rotated facilitating the multi-dose mechanism. This is generally not possible with pre-filled syringes or cartridge base devices as usually the plunger rod is firmly attached to the stopper and unable to rotate relative to the stopper.

The syringe system of the present invention may be supplied to the user in many variants as per the tables below.

| Cartridge | Septum Needle | Injection Needle | Type |
|---|---|---|---|
| N | N | N | Medical Device |
| N | Y | N | Medical Device |
| N | Y | Y | Medical Device |
| Y | N | N | Combination Product |
| Y | Y | N | Combination Product |
| Y | Y | Y | Combination Product |

In addition, each variant above may be supplied with the plunger rod in place or not making a total of 12 variants for use with cartridges.

| Pre-filled syringe | Type of PFS | Type |
|---|---|---|
| N | — | Medical Device |
| Y | Staked Needle | Combination Product |
| Y | Luer | Combination Product |

In addition, each variant above may be supplied with the plunger rod in place or not making a total of 6 variants for use with pre-filled syringes.

The current invention in particular may be considered a multi-dose syringe system which is elegant in its simplicity and which is easy to implement.

Various embodiments of the present invention, by way of example, are provided in the figures. Therein,

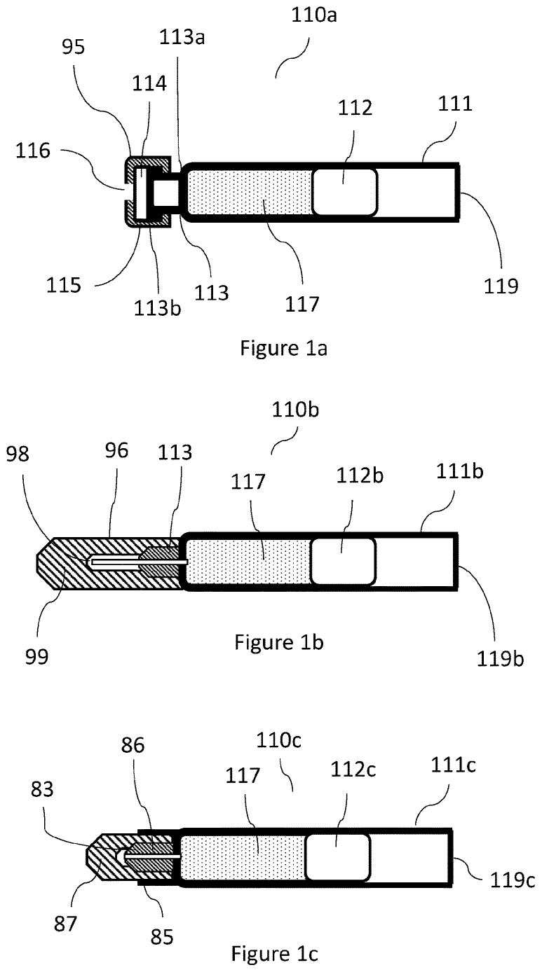

FIG. 1a shows a ‘state of the art’ standard ISO 13926 Cartridge.

FIG. 1b shows a ‘state of the art’ standard ISO 11040 staked needle pre-filled syringe.

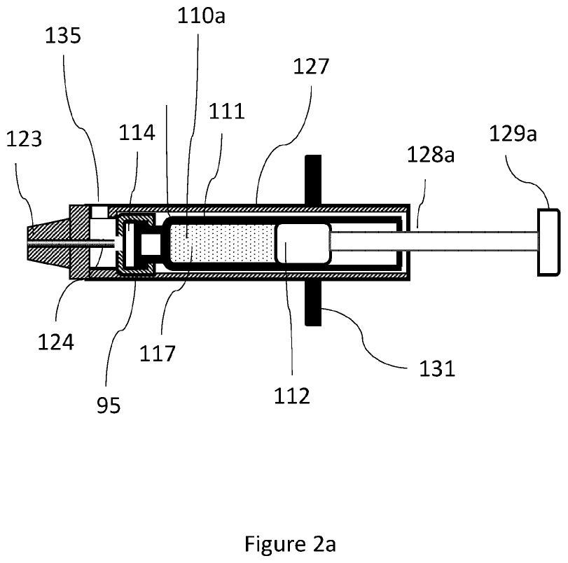

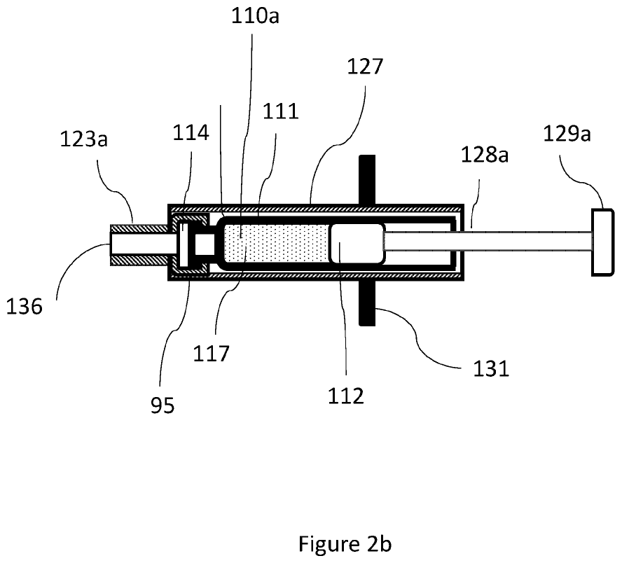

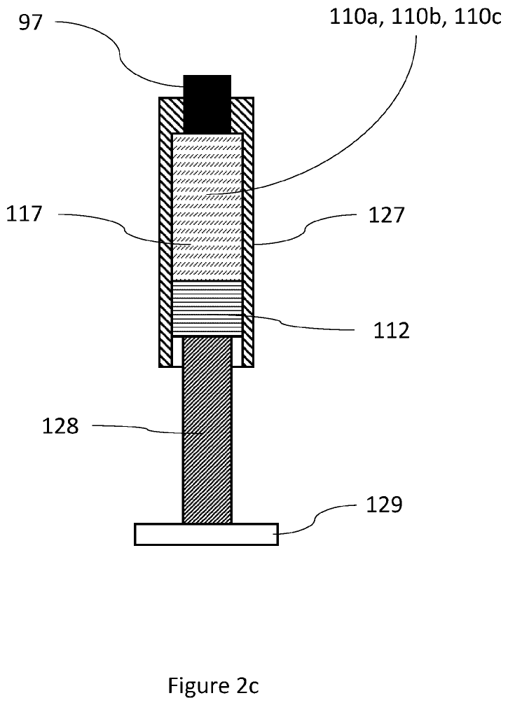

FIG. 2a shows a single dose, cartridge based, syringe generally as per the prior art.

FIG. 2a shows another single dose, cartridge based, syringe generally as per the prior art.

FIG. 2c shows a schematic of the basic components of the present invention.

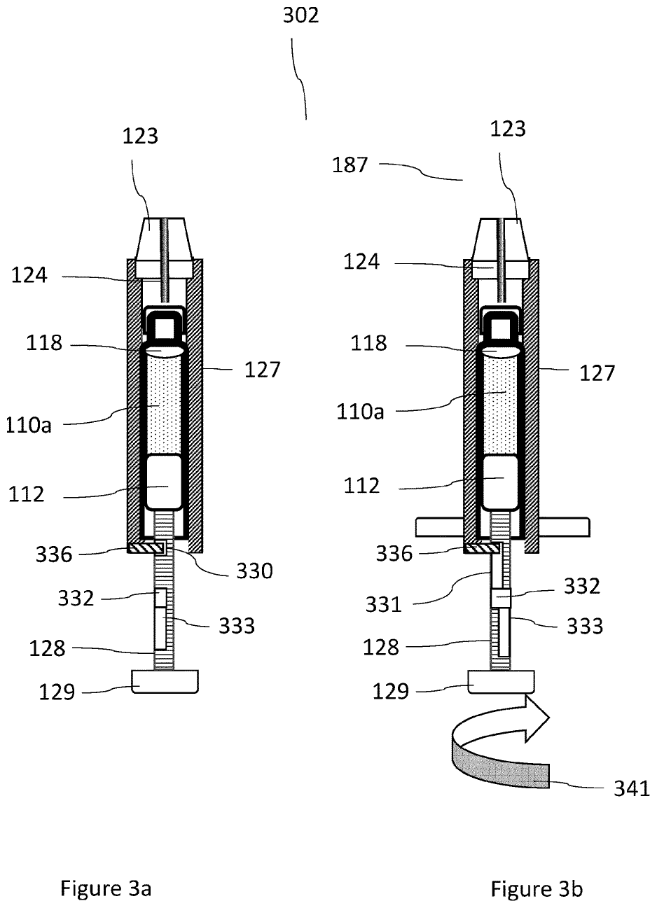

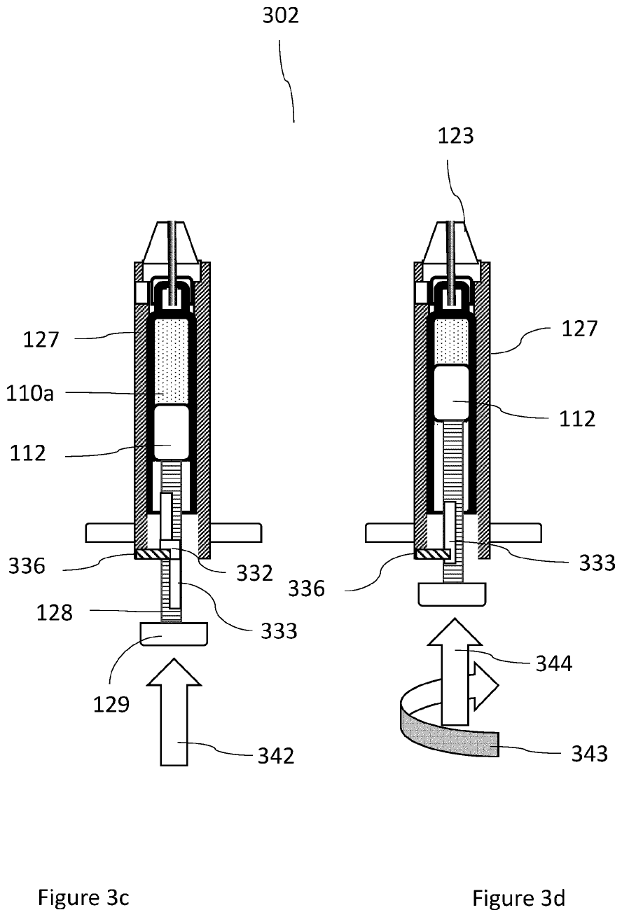

FIG. 3 show the invention where an integrated precise multi dosing mechanism is shown.

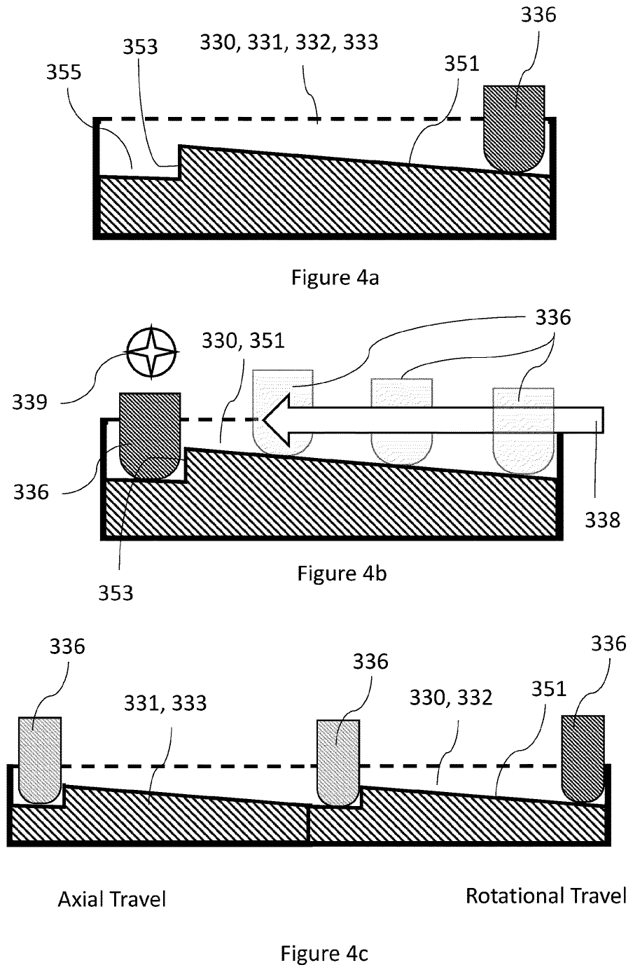

FIGS. 4 and 5 show non-return plunger rod mechanisms.

FIG. 5 show possible ramp depth profiles for the grooves.

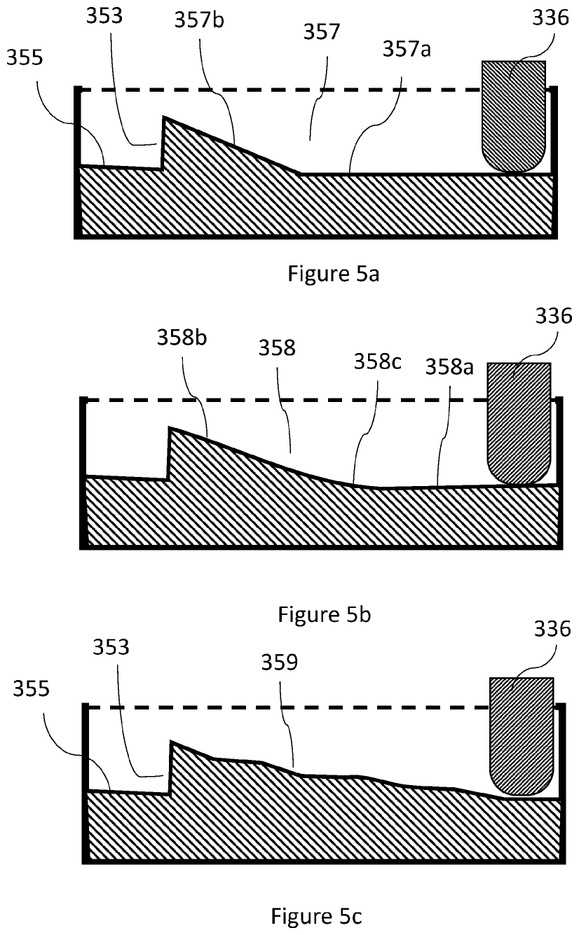

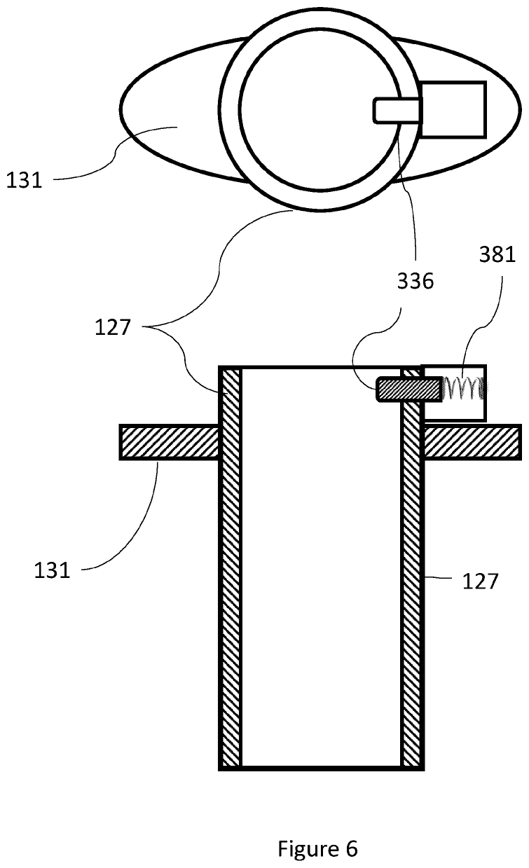

FIG. 6 shows the biased or spring-loaded pin or engagement member.

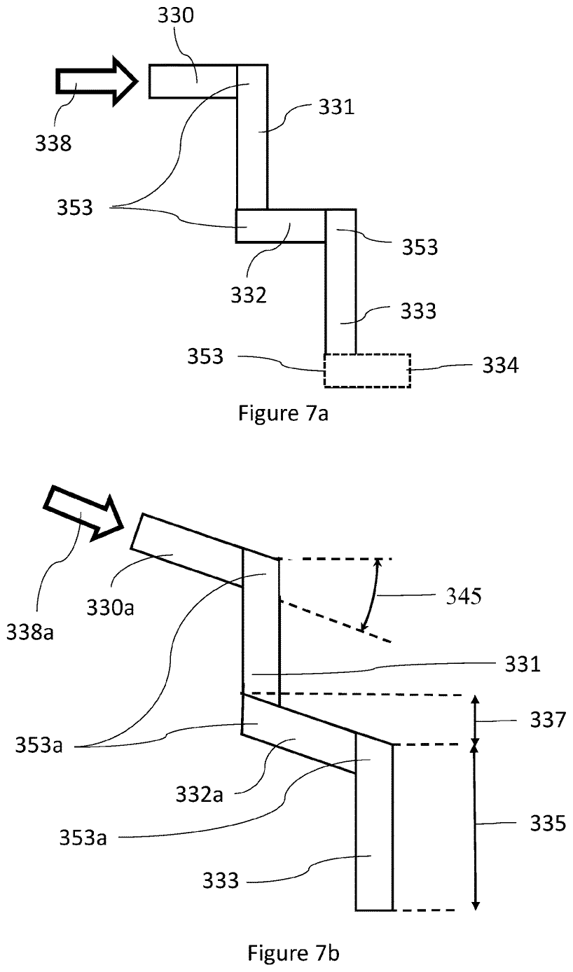

FIG. 7 shows a schematic of the grooves with slopes as per the current invention.

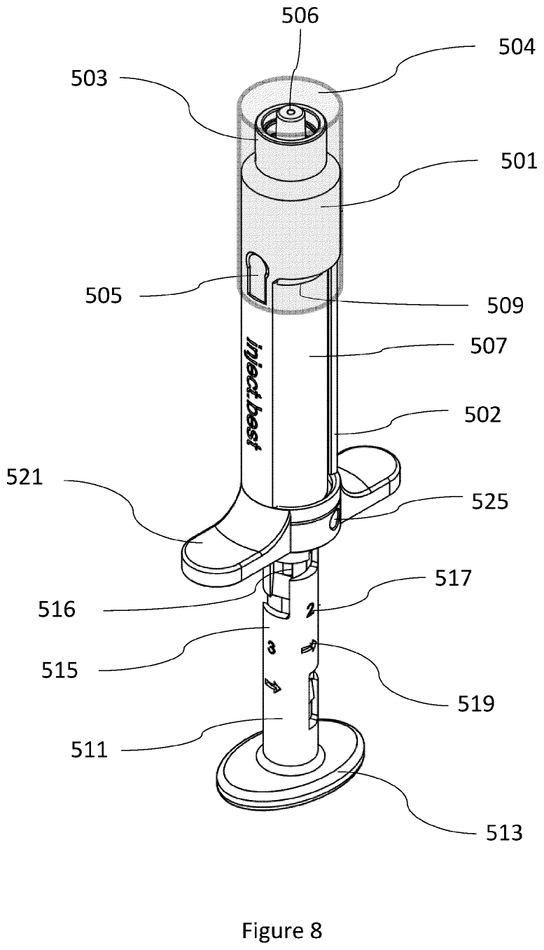

FIG. 8 shows the current invention for use with a Cartridge.

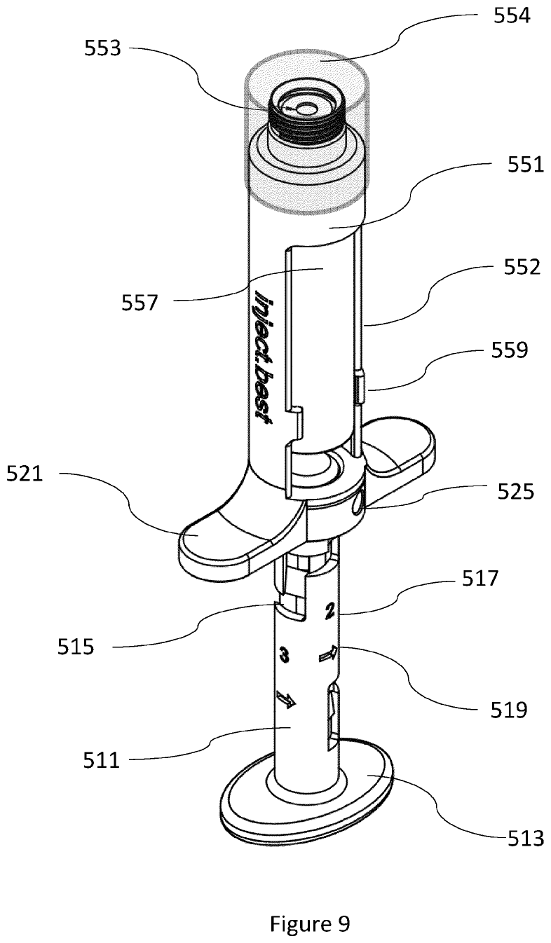

FIG. 9 shows the current invention for use with a Cartridge.

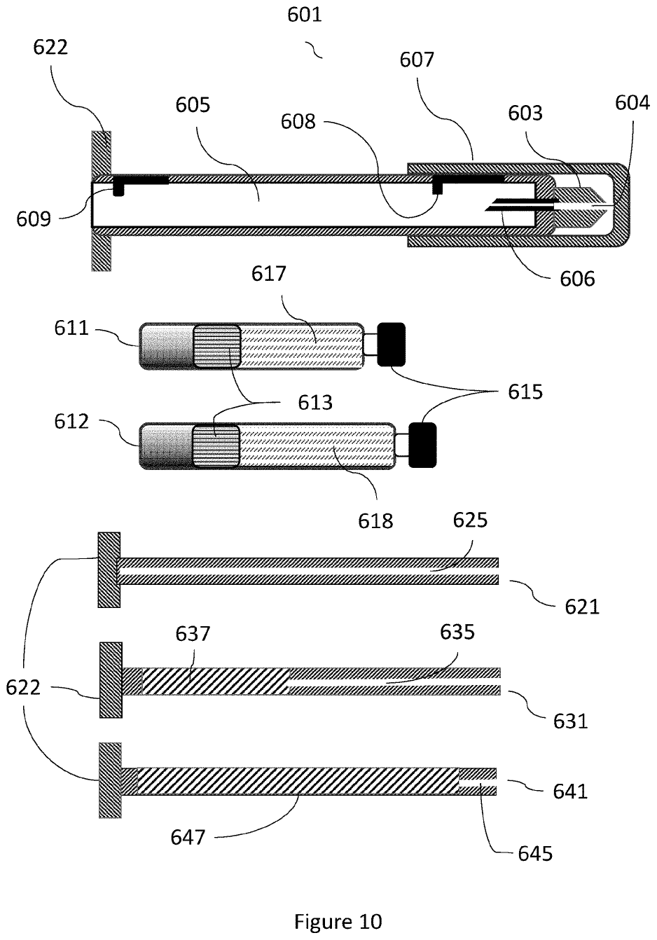

FIG. 10 shows the current invention for use with a Cartridge and various dosing rods.

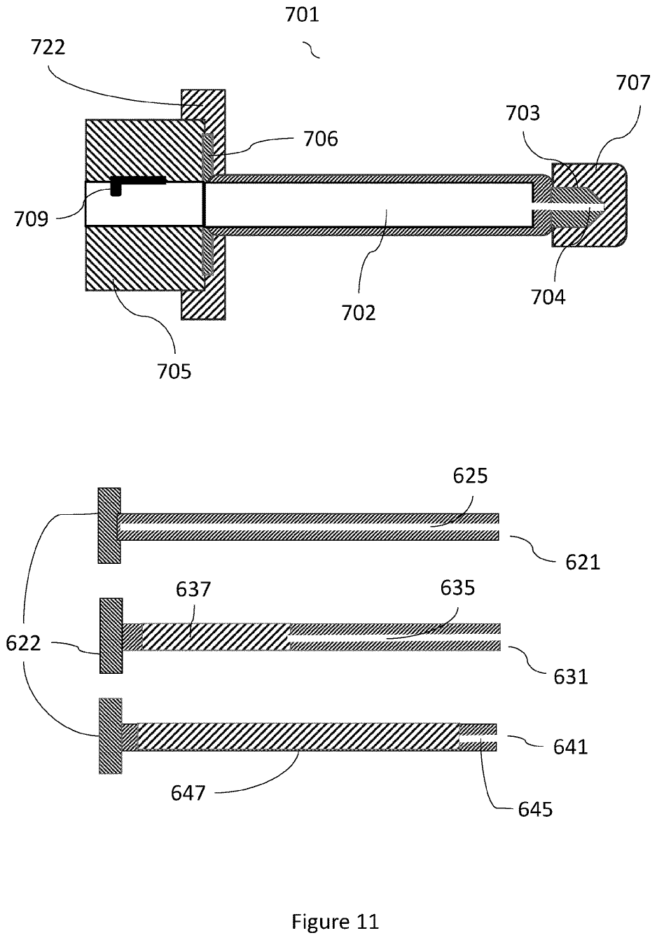

FIG. 11 shows the current invention for use with a PFS and various dosing rods.

The Figures generally show the embodiment with the movable cartridge and fixed septum needle. The multi-dose mechanism is the same for the other embodiments with the fixed cartridge and fixed pre-filled syringe.

Generally identical parts are provided with the same reference numbers in all figures. The invention will be explained below primarily with reference to the embodiments shown. However, other embodiments are of course also conceivable and covered by the present invention.

In FIG. 1a a cartridge 110 has a plastic or glass barrel 111, an open proximal end 119 and a necked 113 and flanged end 113b at a distal end forming a shoulder 113a with the barrel. A movable stopper plunger 112 at open end 119 and a rubber cap assembly 95 at the necked end or distal end 113 seal the drug formulation solution 117 within the barrel 111. Cap assembly 95 has a septum 114 which is held in place by a metal crimp or ferrule 115 which is in turn crimped to the cartridge neck 113. Ferrule 115 has a hole 116 to allow needle insertion through the septum 114.

Such cartridges are fully described in ISO 13926 and can have glass or plastic barrels.

In FIG. 1b a pre-fillable syringe 110b has a plastic or glass barrel 111b an open proximal end 119b and a distal end 113 holding a needle assembly 96 comprising of an injection needle 98 sealed by a shield 99. A movable stopper plunger 112a at open proximal end 119a and the needle shield 99 at the distal end seal the drug formulation solution 117 within the barrel 111b.

In FIG. 1c a pre-fillable syringe 110c has a plastic or glass barrel 111c an open proximal end 119c and a luer cone 83 with passageway 86 and a luer lock thread 85 at the distal end. A movable stopper plunger 112c at open proximal end 119c and a luer cap 87 at the distal end seal the drug formulation solution 117 within the barrel 111c.

FIG. 2a shows a basic single-dose syringe system of the prior art as described in PCT/EP2022/075752. A cartridge 110a is mounted within a casing or housing 127 at a first position. A luer connection 123 is located at the distal end of the syringe housing. A septum needle 124 is firmly held at the front end or distal end of housing 127 and in fluid communication with a luer or other connector 123 at the distal end of the housing 127. Cartridge 110 is axially free to move under user force in the direction of the septum needle 124 to a second position and when axially pushed by pushing plunger rod 128a at pad 129a it forces septum needle 124 to pierce the cartridge cap 95 establishing fluid communication between the cartridge liquid contents and the luer connection 123.

The user can push the plunger rod 128a using the thumb pad 129 and finger flanges 131 in the usual way to axially push the cartridge 110 to a second position to enable the syringe system and prime the system if required and perform the injection of contents 117 via luer connection 123.

Connection 123 may be a luer slip or a luer lock for hypodermic needles or tubing.

Gas-access pathways 135 may be placed in casing 127 to facilitate gas sterilization of the needle assembly.

Cartridge 110 may be locked into place in the first position before use to prevent the cartridge septum being inadvertently pierced by needle 124. Alternatively, cartridge 110 may be held in the first position by friction and for instance by friction at the cartridge shoulder.

FIG. 2a shows the embodiment where the cartridge 110 is movable from a first position to a second position and where the housing 127 has a septum needle 124 firmly attached. Where the cartridge 110 is fixed in position and the user attaches a double ended needle the cartridge is always fixed in the second position and a septum needle is not attached to the housing but there is a passageway to allow the double ended needle through. This is shown in FIG. 2b.

In FIG. 2b the luer connection has been replaced with a screw fitting 123a including a hole or passageway 136 to allow the insertion of a septum needle. Cartridge 110 is fixed into position and unable to move axially. Such an arrangement can be used with state of the art dental and/or insulin double ended needles.

FIG. 2c shows a schematic of the basic parts of the present invention. A housing 127 holds a drug container 110a, 110b or 110c with contents 117. At the distal end there is a drug delivery port 97 and at the proximal end an accurate multi-dosing plunger rod 128 with finger pad 129.

The drug delivery port 97 connects the contents of the drug container 110a, 110b or 110c of FIG. 1 to either an injection needle or a connector such as a luer or a threaded connector for double ended needles. In the case of pre-filled syringes 110b and 110c of FIGS. 1b and 1c the delivery port 97 in in effect the injection needle or luer connector of the syringe. In the case of cartridges 110a of FIG. 1a that have a septum the drug delivery port 97 is formed by the engagement of the septum 114 with an injection needle or luer or other connector via a septum needle.

FIG. 3 shows a multi-dose syringe of the prior art as described in PCT/EP2022/075752. The device shown includes the movable cartridge and fixed septum needle. Device 302 is shown before use in a first position in FIG. 3a. It is shown after the first rotation of the plunger rod in FIGS. 3b. FIG. 3c shows the device after the cartridge has been pushed for the first time and the device has been primed and FIG. 3d shows the device after a first dose has been delivered. The device of FIG. 3 has an accurate dosing mechanism with radial grooves 330 and 332 and axial grooves 331 and 333 plus a biased pin or engagement member 336. The example shown has a priming stroke and only one dose. By adding further grooves to the plunger rod multi-doses can be delivered.

In FIG. 3 spring loaded pin 336 which is attached to body 127 is firstly engaged in slot or radial groove 330 which is formed in plunger rod 128 as shown in FIG. 3a. To operate plunger rod 128 is first rotated until pin 336 is lined up with axial groove 331 as shown by arrow 341 in FIG. 3b. The plunger rod 128 is then pushed by the user as shown by arrow 342 until pin 336 reaches radial groove 332 and cannot move further as shown in FIG. 3c. During this first axial movement of the plunger rod 128 the cartridge 110 moves forward from first position to second position and the needle 124 pierces the cartridge septum and the air bubble 118 (if present) and some liquid contents are expelled from device via luer connector 123 priming the device.

In FIG. 3d the injection has taken place. To get from FIG. 3c to FIG. 3d first the plunger rod 128 is rotated as shown by arrow 343 so that pin 336 travels along radial groove 332 and lines up with axial groove 333. The plunger rod 128 is then pushed as shown in arrow 344. As plunger rod 128 is pushed as shown by arrow 344 it moves until pin 336 reaches the end of slot 333 and cannot move further administering the exact dose of drug formulation determined by the length of axial groove 333.

The plunger rod may have further radial and axial slots so that more than one exact dose of drug can be administered making a multi-dose syringe. With each injection the plunger rod is rotated and then pushed. Before the injection the user may attach a new needle to the luer connector 123. When there is more than one dose more than one needle may be used especially if more than one subject is to be injected.

The first radial 330 slot is optional and is used as a safety feature to prevent premature axial plunger movement or cartridge movement.

Note that the position of the slots and pin or engagement member may be reversed with slots 330, 331, 332 and 333 formed in casing 127 with the pin 336 forming part or attached to plunger rod 128.

If the cartridge is filled without an air or a gas bubble and priming is not required, the first slot 331 can be used only for moving the cartridge forward to the second position ready for injection.

Note that in the above Figures the luer connection might be a luer slip or a luer lock for needle attachments or a line attachment and further may be a catheter tip or an eccentric tip to enable the user to attach intravenous infusion lines. It is also a feature of the present invention that if the syringe system is supplied in a sterile blister or sterile pack a nozzle cap is not essential.

The luer connection 123 can be replaced by a fixed injection needle.

FIGS. 4 to 11 show embodiments of the present invention.

In FIG. 4a the grooves are shown with a depth profile. In FIG. 4a groove 330, 331, 332, 333 has profile including a ramp 351, and a step 353, 355. Pin or engagement member 336 is biased towards ramp 351 and shown at the beginning of radial or axial travel along grooves 330, 331, 332 or 333. In FIG. 4b arrow 338 shows the relative direction of pin 336 and groove 351 where pin or engagement member 336 is shown at the end of its travel having passed step 353. At this position the travel direction of pin 336 cannot be reversed. Cross 339 shows the future direction of travel of pin 336 at ninety degrees to groove 330 and moving from radial groove 330 to an axial groove.

In FIG. 4c two separate grooves are shown. Groove 330, 332 is a radial groove while groove 331, 333 is an axial groove. There is a ninety degree turn in between the two which for simplicity is not shown in FIG. 4. In FIG. 4 radial groove 330 is shown opened up and flattened for clarity.

There may be two or more axial and radial grooves a pair for each dose.

FIG. 5 show alternative ramp profiles. In FIG. 5a the profile 357 starts as a horizontal 357a and then moves to a ramp 357b towards step 353, 355. In FIG. 5b profile 358 starts as a horizontal 358a then blends into ramp 358b via a gentle curve 358c.

In FIG. 5c the ramp 359 has a random profile but generally ramping up towards step 353, 355.

One further benefit or improvement of having the grooves with a profile combined with a biased pin or engagement member is that it gives feedback to the user by indicating the plunger rod is in the correct position before and after rotating or pushing it.

FIG. 6 shows a spring loaded or biased engagement member 336 spring biased by coiled spring 381. Pin 381 and coiled spring 381 can be formed or molded as part of housing 127 in a single part.

FIG. 7a shows a schematic of the grooves where grooves 330, 332 and 334 are radial grooves and grooves 331 and 333 are axial groves. Arrow 338 denotes the relative direction of the pin or engagement member. The length of axial grooves determines the dose size for any given diameter of drug container. The length of the radial grooves determines the rotational angle in between axial grooves.

FIG. 7b shows a schematic of the grooves where grooves 330a and 332a are radial grooves and grooves 331a and 333a are axial groves. Arrow 338a denotes the relative direction of the pin or engagement member. The length 335 of axial grooves determines the dose size for any given diameter of drug container. The radial grooves 330a and 332a have in addition an axial component which is determined by groove angle 345 and axial length 337. The axial length 337 determines the amount of drug formulation expelled from the syringe during the rotation of the plunger rod. This can be used to re-prime the syringe after changing the injection needle.

FIG. 8 shows a complete assembly of the invention. A housing 501 has a slot 507 for radially inserting a cartridge. Slot 502 also serves as a viewing window. Housing 501 has a finger flange 521 at the proximal end and a luer connector 503 at the distal end. Cartridge 507 is in a first position held by snaps 505 at its shoulder 509. A luer cap 504, shown transparently for ease of viewing, holds the shoulder snaps 505 in place preventing them opening. A septum hypodermic needle, not shown, is rigidly held in the housing 501 with the needle sharp end pointing towards the cartridge 507 septum.

A multi-dose plunger rod 511 is attached at the proximal end having a series of axial grooves 515 and radial grooves 516. All grooves have ramps and steps as described in FIGS. 4 and 5 to prevent reverse travel and for user finger feedback. A pin 525 on housing 501 engages with slots 515 and 516 to control plunger rod movement. Arrows 519 and numbers 517 show through a viewing window, placed opposite pin 525 and not shown on FIG. 8, to indicate dose number and next operation. To operate the luer cap 504 is removed and the plunger rod 511 is pushed to move the cartridge 507 past the snaps 505 and towards the septum needle which perforates the cartridge septum connecting liquid contents of cartridge 507 with luer exit hole 506 and priming the device by expelling any gas bubbles in the cartridge 507. A hypodermic needle may be connected to the luer 503 before the plunger rod is first pushed.

FIG. 9 shows another complete assembly of the invention. Housing 551 holds a cartridge 557 in a fixed position where cartridge is inserted via slot 552. Tabs 559 prevent cartridge moving radially once inserted allowing for a larger slot 552. A treaded needle connected is disposed at the distal end of housing 551 and a finger flange 521 at the proximal end. Plunger rod 511 has features as described in FIG. 8. To operate the user attaches as double ended needle to the threaded connector 553 by first removing optional dust cap 554. Double ended needle comprises of a treaded body and injection and septum needles. When needle is fully screwed in the septum needle perforates the cartridge septum allowing operation of the syringe system.

FIG. 10 shows yet another embodiment of the invention. A housing 601 is designed to accommodate cartridges 611 and 612 of any length in space 605. Cartridges 611 and 612 have septum caps 615 and respectively fill volumes 617 and 618 sealed by a movable stopper 613. Housing 601 has a finger flange 622 at the proximal end and a luer connector with liquid passageway 604 at the distal end connected to a septum needle 606. A snap 608 holds the cartridge at the shoulder in a first position after it is inserted and the snaps 608 are prevented from opening and cartridge moving to a second position until the cap 607 is removed. A spring-loaded pin 609 engages with the plunger rod when inserted.

Various dosing rods may be used with the device all having a thumb pad 622. Rod 621 is a single dose rod that is inserted by engaging groove 625 with pin 609 of housing 601. Rod 631 is a single dosing rod with a groove arrangement 637 to allow for a priming dose and an accurate injection dose. Rod 631 is inserted into housing 601 by engaging groove 635 with pin 609 of housing 601. Rod 641 is a multi-dose rod for multiple doses and if required a priming dose with groove arrangement 647. Rod 641 is inserted into housing 601 by engaging groove 645 with pin 609 of housing 601.

FIG. 11 shows yet another embodiment of the invention. A housing 701 is designed to accommodate pre-filled syringe 702 of any length. Pre-filled syringe 702 is attached to housing 705 by its flange 706. Pre-filled syringe 702 may be a staked needle pre-filled syringe or a luer pre-filled syringe. Housing 705 has a finger flange 722 which also holds the syringe flange 706 in place. A spring-loaded pin 709 engages with the plunger rod when inserted.

Various dosing rods may be used with the device all having a thumb pad 622. Rod 621 is a single dose rod that is inserted by engaging groove 625 with pin 709 of housing 705. Rod 631 is a single dosing rod with a groove arrangement 637 to allow for a priming dose and an accurate injection dose. Rod 631 is inserted into housing 705 by engaging groove 635 with pin 709 of housing 705. Rod 641 is a multi-dose rod for multiple doses and if required a priming dose with groove arrangement 647. Rod 641 is inserted into housing 705 by engaging groove 645 with pin 709 of housing 705.

Note that that in FIGS. 10 and 11 rods 621, 631 and 641 may have grooves with a depth profile with ramps and steps and be non-reversible. Note also that rods 621, 631 and 641 may have a further radial groove after the initial entry guide groove so that the rod may be rotated and locked into place before starting priming/dosing.

1. A syringe comprising:
an outer casing or syringe housing with finger holds at the proximal end and a drug delivery port at the distal end capable of firmly holding;
a drug container having a generally tubular glass or plastic barrel with drug delivery means at the distal end of the barrel and a stopper/piston disposed close to the proximal end of the barrel to contain the medicament or other formulation between the stopper/piston and the drug delivery means,
where the drug delivery means engages with or forms a drug delivery port at the distal end of the syringe housing,
a plunger rod with finger pad at one end and in communication with the cartridge stopper/piston at the other end,
where the ensemble of plunger rod and syringe housing has a guiding and engagement system comprising one or more radial grooves and one or more axial grooves in communication with one another provided at one of plunger rod and syringe housing and in engagement with a pin or engagement member connected to the other of plunger rod and the syringe housing, respectively, where the pin travels relative to the grooves as the user rotates and pushes the plunger rod to prime or deliver one or more precise doses of liquid formulation via the drug delivery port,
where the plunger rod is free to rotate relative to the container stopper and where the grooves have a profile that prevents the user reversing the radial movement and/or the axial movement of the plunger rod.

2. A syringe according to claim 1 wherein the guiding and engagement system comprises at least two axial grooves.

3. A syringe according to claim 1 where the pin is biased against the grooves.

4. A syringe according to claim 1 where the drug container is a cartridge with a pierceable septum or cap where a septum needle is rigidly held at the distal end of the syringe housing and is in fluid communication with a luer slip or luer lock with means to connect an injection needle or a transfer line and where the cartridge is held in a first position and is prevented from substantially moving unaided axially forwards within the syringe housing,
where during the first axial movement of the plunger rod the cartridge is forced forwards relative to the septum needle and into a second position when the septum needle perforates the cartridge septum establishing fluid communication between the cartridge contents and the luer connector and the injection needle or line.

5. A syringe according to claim 1 where the drug container is a cartridge with a pierceable septum or cap where a septum needle is rigidly held at the distal end of the syringe housing and is in fluid communication with an injection needle projecting from the distal end of the syringe housing and where the cartridge is held in a first position and is prevented from substantially moving unaided axially forwards within the syringe housing,
where during the first axial movement of the plunger rod the cartridge is forced forwards relative to the septum needle and into a second position when the septum needle perforates the cartridge establishing fluid communication between the cartridge contents and the injection needle.

6. A syringe according to claim 1 where the drug container is a cartridge with a pierceable septum or cap where a threaded or other fitting projects from the distal end of the syringe housing with means to connect an injection needle and septum needle combination, and where the cartridge is held axially in position,
where once the needle combination is attached to the syringe housing the septum needle perforates the cartridge establishing fluid communication between the cartridge contents and the injection needle.

7. A syringe according to claim 1 where the drug container is a staked needle pre-filled syringe and where the staked needle pre-filled syringe is held axially in position in the syringe housing and where the staked needle and needle shield combination project from the distal end of the syringe housing.

8. A syringe according to claim 1 where the drug container is a pre-filled syringe with a luer connection and where the pre-filled syringe is held axially in position in the syringe housing where the luer slip or luer lock and cap project from the distal end of the syringe housing, and where an injection needle or line can be attached to the luer connector.

9. A syringe according to claim 1 where the syringe is a multi-dose syringe.

10. A syringe according to claim 1 where the syringe or the syringe and drug container combination are terminally sterilized inside a blister.

11. A syringe according to claim 1 where the syringe has a dose counter.

12. A syringe according to claim 1 where the syringe is re-usable and may be used with more than one drug container.
